(19) United States
(12) Patent Application Publication
(10) Pub. No.: US 2008/0154175 A1
(43) Pub. Date: Jun. 26, 2008

(54) Novel Tampon Applicator

(76) Inventors: David J. Chase, Somerville, NJ (US); Tara Glasgow, New Hope, PA (US); Raymond J. Hull Jr., Hampton, NJ (US); Stephen J. Mohary, Pennington, NJ (US); Linda M. Pierson, Stockton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/817,191

(22) PCT Filed: Mar. 30, 2006
(86) PCT No.: PCT/US2006/012574

§ 371 Date: Aug. 27, 2007

Related U.S. Application Data
(60) Provisional application No. 60/667,337, filed on Mar. 31, 2005.

(51) Int. Cl. A61F 13/20; A61B 19/02

(52) U.S. Cl. 604/12; 604/14; 604/15; 206/438

(57) ABSTRACT

An applicator for delivering an insertable device into a body cavity includes (a) a barrel having an insertion end and an opposed gripper end, (b) a lubricant carrier operatively linked to the barrel (c) a fluid lubricant in fluid communication with lubricant carrier, and optionally (d) a plunger arranged and configured to be moveable within the barrel to expel the insertable device from the barrel and into a body cavity. The barrel is arranged and configured to contain the insertable device. The applicator may be included in a packaged applicator system.

### CROSS-REFERENCE TO RELATED APPLICATIONS

This invention is related to the following copending application: U.S. Ser. No. 11/______, filed on even date herewith, entitled “Lubricated Tampon Applicator” (Att'y Docket, J&J-5169).

### FIELD OF THE INVENTION

The present invention relates to applicators for delivering materials into mammalian body cavities. The applicator is particularly useful for delivering intravaginal devices, such as catamenial devices, into a vaginal canal.

### BACKGROUND OF THE INVENTION

Applicators for delivering materials into a body cavity typically comprise a tubular insertion member having an insertion end and a gripper end opposite thereof, and an elongate expulsion member slideably fitted within the tubular insertion member for expelling the contained materials.

### SUMMARY OF THE INVENTION

The present invention relates to an applicator for delivering an insertable device into a body cavity. In one embodiment, the applicator includes (a) a barrel having an insertion end and an opposed gripper end, (b) a lubricant carrier operatively linked to the barrel (c) a fluid lubricant in fluid communication with lubricant carrier, and optionally (d) a plunger arranged and configured to be moveable within the barrel to expel the insertable device from the barrel and into a body cavity. The barrel is arranged and configured to contain the insertable device.

The lubricant carrier may be textured. It may be affixed to the barrel or to the insertable device. The lubricant carrier may include at least one flexible element having an attachment portion that is affixed to the barrel, and a distal portion that is capable of extending away from the barrel. It may also be formed of a plurality of flexible elements, each flexible element having at least one attachment portion that is affixed to the barrel, and at least one distal portion that is capable of extending away from the barrel. In such case the at least one attachment portion is affixed to the insertion end of the barrel.

The lubricant carrier may have a fluid lubricant is disposed thereon, or a fluid lubricant may be contained in at least one reservoir capable of being in fluid communication with the carrier. In one embodiment, the fluid lubricant substantially saturates lubricant carrier.

An alternative embodiment relates to a packaged applicator system. In this embodiment, the system includes (a) an insertable device for delivery into a body cavity contained within (b) a barrel having an insertion end and an opposed gripper end; (c) lubricant carrier affixed to the barrel; (d) a fluid lubricant disposed on lubricant carrier; and optionally (e) a plunger arranged and configured to be moveable within the barrel to expel the insertable device from the barrel and into a body cavity. The applicator is contained within an overwrap package including a first package element arranged and configured to isolate the lubricant (and moisture derived therefrom) from the insertable device and a second package element enclosing the applicator. Again, the barrel is arranged and configured to contain the insertable device. In a variation, the packaged applicator system may have the first package element arranged and configured to sealingly contain a fluid lubricant.

Another embodiment relates to a packaged applicator including (a) an insertable device for delivery into a body cavity contained within (b) a barrel having an insertion end and an opposed gripper end; (c) a lubricant carrier affixed to the barrel; (d) a fluid lubricant contained in reservoir in fluid communication with lubricant carrier; and optionally, (e) a plunger arranged and configured to be moveable within the barrel to expel the insertable device from the barrel and into a body cavity. The applicator is enclosed in a package element.

### BRIEF DESCRIPTION OF THE DRAWING

FIG. 1A is a perspective view of an applicator according to the present invention comprising a tubular insertion member and a lubricant carrier disposed thereon.

FIG. 1B is a cross-section along line B-B of FIG. 1A.

FIG. 2 is a longitudinal cross-section of an applicator, such as depicted in FIG. 1A, having a textured outer surface that imparts a texture to a lubricant carrier affixed thereto.

FIG. 3 is a side elevation of an alternative embodiment of the applicator of the present invention having a lubricant carrier formed of a plurality of flexible elements.

FIG. 4 is a partially cut-away side elevation of an alternative embodiment of the applicator of the present invention having a lubricant carrier having at least one attachment portion affixed to the insertable device contained within the applicator.

FIGS. 5A and 5B are partially cut-away side elevations of an alternative embodiment of the applicator of the present invention having a lubricant carrier having at least one attachment portion affixed to the inside surface of the insertion member.

FIG. 6A is a side elevation of an alternative embodiment of the applicator of the present invention having a lubricant carrier having at least one attachment portion affixed to the outer surface of the insertion member.

FIG. 6B is a longitudinal cross-section of the embodiment of FIG. 6A showing the lubricant carrier in a semi-inverted arrangement.

FIG. 7 is a side elevation of an applicator of the present invention packaged in a manner to separate the lubricant carrier from the insertable device.

### DETAILED DESCRIPTION OF THE INVENTION

As used in the specification and claims, the term “diameter” as used in herein the specification and the claims means a chord passing through the center of a figure or body; the length of a straight line through the center of an object

As used herein the specification and the claims, the term “intravaginal device” and related terms includes support devices, obstructing devices useful to block the flow of and/or collect bodily liquids, and the like. The term includes, without limitation, incontinence devices and vaginal supports, such as pessaries; and obstructing devices, such as menstrual collection cups and inflatable or expandable vaginal blocking devices (devices which do not, themselves, absorb the bodily liquids).

While the present invention generally relates to applicator devices having a tubular insertion member, the following detailed description will refer, specifically, to a tampon applicator for ease of understanding. One of ordinary skill in the art will recognize other uses for this invention including, without limitation, applicators for other catamenial devices, such as collection cups, inflatable plugs or cups, and the like; incontinence devices; suppositories; nasal packing; and the like.

The present invention relates to applicators for delivering materials into body cavities, comprising an elongate insertion member for at least partial insertion into a body cavity. The elongate insertion member has an insertion end and gripper end opposite thereof.

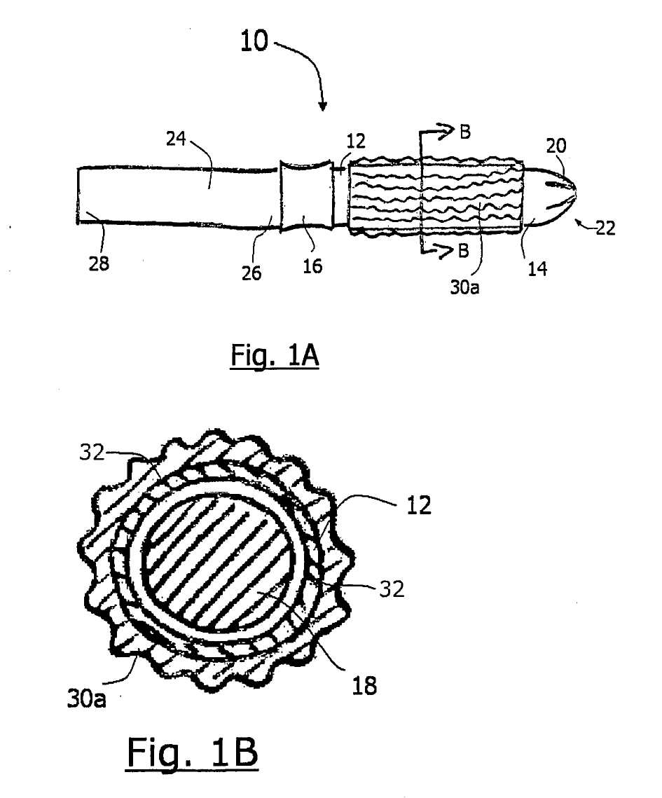

Referring now to the drawings, wherein like reference numerals designate like elements, FIG. 1 depicts an applicator 10, comprising a tubular insertion member 12, having an insertion end 14 and a gripper end 16 that is suitable to contain an insertable device 18 for the body cavity. The insertion end 14 may have a plurality of inwardly curved petals 20 that form a substantially closed feature 22, and the gripper end 16 may have a structure 17 to enhance the user's grip on the applicator. The applicator 10 also includes an expulsion member 24 having a first end 26 that is insertable into the tubular insertion member 12 and is capable of bearing against the device 18. The expulsion member 24 terminates in a second end 28, opposite the first end 26, that may be manipulated to move the first end 26 within the tubular insertion member 12. The first end 26 of the expulsion member 24 is arranged and configured for slideable introduction into the tubular insertion member 12 through its gripper end 16.

The applicators 10 or other tubular devices of the present invention can have tube geometries or cross-sections that are useful to contain the object to be inserted. Often, the shape of the insertable device 18 contained suggests the shape of the tubular insertion member 12, but departures from this general rule may be made. Therefore, the tubular insertion member 12 may take on numerous cross-sectional shapes including, without limitation, circular, oval, polygonal (e.g., trapezoidal, rectangular, triangular), and the like. For example, cylindrical tampons may be contained within rectangular insertion members and trapezoidal tampons (such as those disclosed in Van Iten et al., U.S. Pat. No. 5,350,371) and cup-shaped tampons (such as those disclosed in Bailey, U.S. Pat. No. 2,330,257) can be contained in a generally cylindrical insertion member. In addition, the insertion member 12 can substantially elongated, curved, or flexible, or it can take on other shapes that are apparent to one of ordinary skill in the art. The specific geometry, itself, is not critical to the practice of the present invention. In addition, the edge of the tubular device (both finished and unfinished) may be a standard, planar edge coincident with a plane perpendicular to the longitudinal axis of the tubular device.

The applicator devices of the present invention can be made of materials known to those of ordinary skill in the art. Generally, the applicators may be plastic or paper. Plastic materials include, without limitation, polyolefins such as polyethylene and polypropylene, polyesters such as polyethylene terephthalate, polycaprolactone, polyvinyl alcohol, ethylene-vinyl acetate copolymers, cellophane, PHBV such as those disclosed in Dabi et al., U.S. Pat. No. 5,910,520 (herein incorporated by reference), starch-based polymers including those disclosed in Dabi et al., U.S. Pat. No. 5,910,520, and the like. The expulsion member can be formed as a solid or a tubular element.

Paper materials include, without limitation, paperboard, cardboard, cup stock, paper, and the like. The paper may be a single layer of material, or it can be a plurality of laminated layers to provide multiple benefits relating to the various layers. Laminated paper material may include a surface layer or coating of plastic, wax, silicone, lubricants, and the like, which may be useful to increase the comfort to the user during insertion and withdrawal. The plastic coating may include, without limitation, those plastic materials listed above. Laminated paper material may also include additional layers such as adhesive layers, tie layers, and the like.

An example of such a surface layer is disclosed in Blanchard, U.S. Pat. No. 6,171,426. A representative, non-limiting list of useful materials to be used as the surface layer includes, waxes, cellophane, polyolefins, polyesters, epoxies, and the like. The surface layers may also include thermal stabilizers, pigments, fragrances, surfactants, antimicrobial agents, medicaments, and the like.

Typical dimensions for each of the tubular insertion and expulsion members include a length of from about 50 to about 100 millimeters, a diameter of from about 8 to about 16 millimeters, and a thickness of from about 0.4 to about 0.6 millimeters. Preferably, the diameter of the expulsion member is less than the diameter of the tubular insertion member to allow for a telescopic arrangement of the two.

The tubular insertion member of the applicator provided by the present invention is preferably substantially closed prior to expulsion of the materials contained therein. Alternatively, the insertion end of the applicator can be more or less open, that is the diameter along the length of the tubular insertion member is substantially equivalent to the diameter of the insertion end. Procter & Gamble, of Cincinnati, Ohio, currently offers for sale an open-ended tampon applicator under the trade name TAMPAX® flushable applicator tampons.

In one set of embodiments, a lubricant carrier (generically “30”) is operatively linked to the applicator. As used herein the Specification and Claims, the term “operatively linked” relates to elements that are connected directly or indirectly to each other. For example, a lubricant carrier affixed to the applicator would be operatively linked. Alternatively, a lubricant carrier that is not physically attached to the applicator, but which is attached to an insertable device contained within the applicator would also be operatively linked to the applicator.

Referring again to FIGS. 1A and 1B, the lubricant carrier 30a can be a textured pile fabric, such as terry cloth, or other textured fibrous structure. In this embodiment, the lubricant carrier 30a is affixed to the tubular insertion member 12 about its outer surface 32. As shown in FIG. 1A, the lubricant carrier 30a extends from the insertion end to the gripper end (as shown, both the extreme insertion and gripper ends are left uncovered by the lubricant carrier 30a).

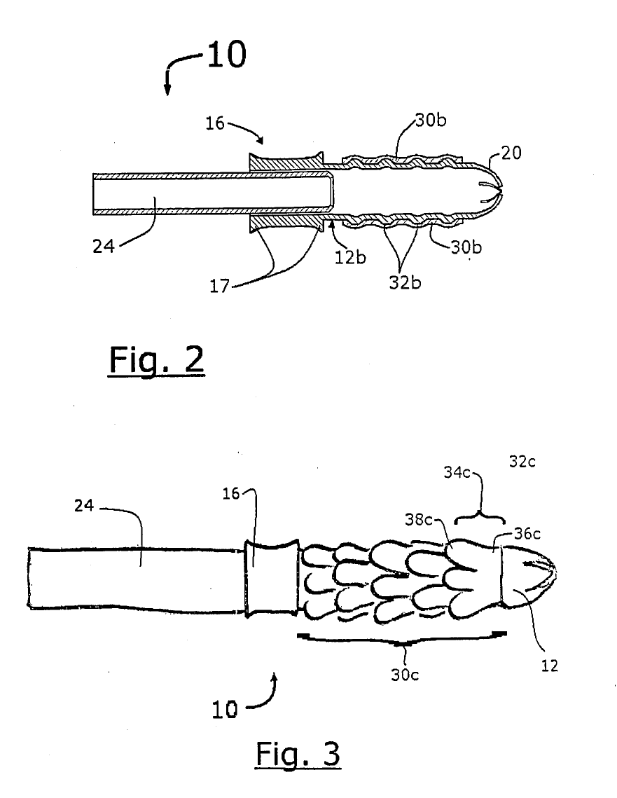

An alternate embodiment is shown in FIG. 2. In this embodiment, the tubular insertion member 12b has a textured outer surface 32b. This textured outer surface 32b imparts a texture to the lubricant carrier 30b affixed thereto. As in the embodiment of FIG. 1A, the lubricant carrier 30b of the present embodiment extends from the insertion end to the gripper end (as shown, both the extreme insertion and gripper ends are left uncovered by the lubricant carrier 30b).

The flexible elements 34 generally also allow the lubricant carrier 30 to “swab” the surfaces of the body cavity into which it is inserted, during both insertion and withdrawal motions. In embodiments in which the flexible elements 34 are only partially attached, they may invert to present a relatively clean swabbing surface during withdrawal. This wiping or cleansing action is helpful to provide a clean and fresh feel to the user. Therefore, lubricant may also incorporate additives. A representative, non-limiting list of such additives includes cleansers, emollients, anti-inflammatories, probiotics, antibiotics, and the like. Specific compositions may include health-promoting compositions such as glyceryl monolaurate as disclosed in Brown-Skrobot, U.S. Pat. No. 5,641,503, the disclosure of which is herein incorporated by reference.

An alternate embodiment incorporating such a “swabbing” functionality is shown in FIG. 3. In this embodiment, the lubricant carrier 30c includes a plurality of flexible elements 34c. Each flexible element 34c has at least one attachment portion 36c that is affixed to the tubular insertion member 12, and at least one distal portion 38c that is capable of extending away from the tubular insertion member 12. This arrangement of the flexible elements 34c permits the lubricant carrier 30c to “swab” the surfaces of the body cavity into which it is inserted, during both insertion and withdrawal motions. Indeed, the flexible elements 34c can invert to present a relatively clean swabbing surface during withdrawal.

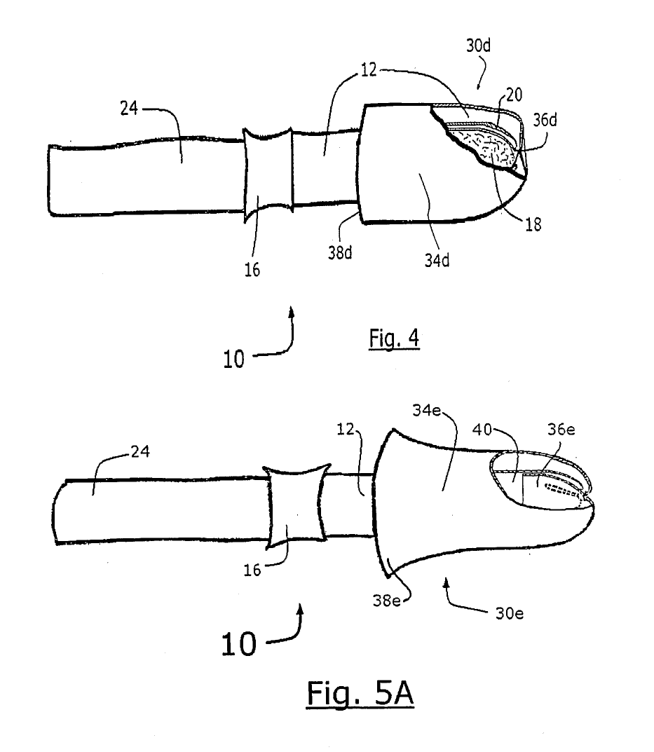

A further alternate embodiment is shown in FIG. 4. In this embodiment, the lubricant carrier 30d includes at least one flexible element 34d that has at least one attachment portion 36d. However, in this embodiment, the attachment portion 36d is affixed to the insertable device 18. Again, the lubricant carrier 30d has at least one distal portion 38d that is capable of extending away from the insertable device 18. This arrangement of the flexible element(s) 34d permits the lubricant carrier 30d to “swab” the surfaces of the body cavity into which it is inserted, during both insertion and withdrawal motions. Again, the flexible elements 34d can invert to present a relatively clean swabbing surface during withdrawal.

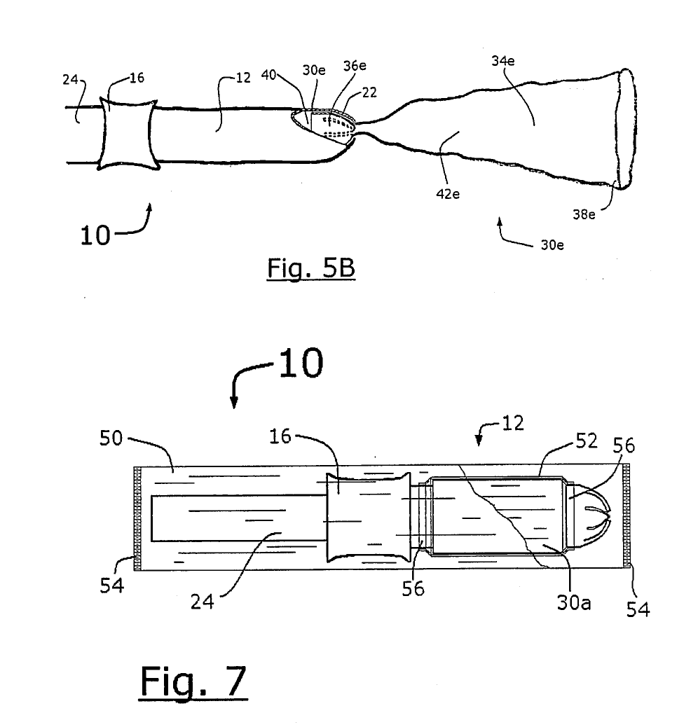

A similar, alternate embodiment is shown in FIGS. 5A & 5B. In this embodiment, the lubricant carrier-30e includes at least one flexible element 34e that has at least one attachment portion 36e. In this embodiment, the attachment portion 36e is affixed to the inner surface 40 of the insertion member 12. Again, the lubricant carrier 30e has at least one distal portion 38e that is capable of extending away from the insertion member 12. This arrangement of the flexible element(s) 34e permits the lubricant carrier 30e to “swab” the surfaces of the body cavity into which it is inserted, during both insertion and withdrawal motions. Again, the flexible elements 34e can invert as shown in FIG. 5B to present a relatively clean swabbing surface 42e during withdrawal.

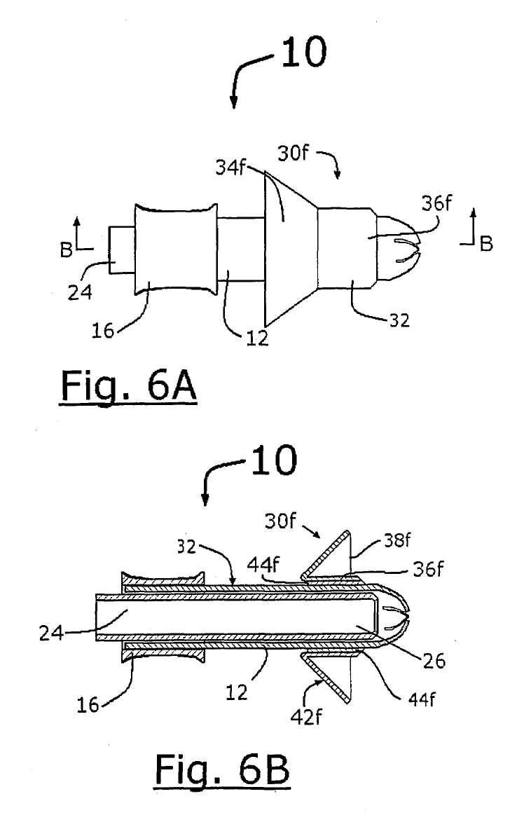

Another alternate embodiment is shown in FIGS. 6A & 6B. In this embodiment, the lubricant carrier 30f includes at least one flexible element 34f that has at least one attachment portion 36f. In this embodiment, the attachment portion 36f is affixed to the outer surface 32 of the insertion member 12. As shown, an adhesive 44f connects the attachment portion 36f to the outer surface 32 of the insertion member 12. One of ordinary skill will recognize that adhesives are only one of a number of possible means for attaching the attachment portion to the insertion member. Again, the lubricant carrier 30f has at least one distal portion 38f that is capable of extending away from the insertion member 12, permitting it to “swab” the surfaces of the body cavity into which it is inserted, during both insertion and withdrawal motions. Again, the flexible elements 32f can invert as shown in FIG. 6B to present a relatively clean swabbing surface 42f during withdrawal.

A fluid lubricant 46 can be included in fluid communication with the lubricant carrier 30. In the embodiments described above, the applicator 10 can be packaged with a fluid lubricant 46 disposed upon and/or within the lubricant carrier 30 (e.g., permeating the carrier), or it can be maintained in a reservoir 48 until needed. Referring to FIG. 7, the applicator, e.g., of FIG. 1, is packaged with a fluid lubricant permeating the lubricant carrier 30a. In this embodiment, the packaging may include both an overwrap package 50 and a lubricant carrier container 52. The overwrap package 50 may include the whole applicator product and may be closed with a pair of end seams 54. The lubricant carrier container 52 is shown being sealingly attached to the insertion member 12 (e.g., at 56) adjacent the lubricant carrier 30a. The packaging of this particular embodiment is intended to separate the fluid lubricant 46 and any possible moisture released therefrom from migrating to the insertable device contained within the applicator 10. Thus, the ends of the lubricant carrier container 52 are preferably sealed to the insertion member 12. Alternatively, the insertable device may be sealed by itself with a barrier material capable of preventing moisture from migrating therethrough.

In alternative embodiments in which the fluid lubricant 46 is not disposed on or within the lubricant carrier 30, the fluid lubricant 46 may be contained within a reservoir 48. Referring to FIG. 8, a reservoir 48g, e.g., a squeeze bulb, is connected to the insertion member 12 through a tube 58. The insertion member 12g has formed in the walls thereof a conduit 60 and manifold 62 that distributes the fluid lubricant through a plurality of apertures 64 formed in the outer surface 32g of the insertion member 12. The fluid lubricant then may permeate the fluid carrier.

The applicator of the present invention can be constructed of paperboard or plastic. The paperboard applicators can be constructed from a single layer of paper material, or from a plurality of laminated layers to provide multiple benefits relating to the various layers. The applicators can be made from sheets of material using several processing including, without limitation: spiral winding as disclosed in Campion et al., U.S. Pat. No. 5,346,468, convolute winding as disclosed in Whitehead, U.S. Pat. No. 4,508,531, and forming a sheet around a mandrel and then sealing an overlapped seam as disclosed in Hinzmann, U.S. Pat. No. 4,755,164.

If the applicator includes a surface layer, the layer may be applied using any useful technique. Many techniques are known for applying the surface layers. A representative, non-limiting list of such techniques includes spraying, extruding, slot-coating, brushing, transfer coating, and the like. Additional processing steps may be required to cure the surface treatments to a useable form other than simple air curing, such as applying irradiation or other forms of energy.

The insertion member of the applicator provided by the present invention is preferably substantially closed prior to expulsion of the materials contained therein. One technique for substantially closing. the insertion end of the applicator is by employing a plurality of inwardly curved petals. The petals will flex and/or hinge to an open position upon expelling materials contained by the applicator. The number of petals generally ranges from about 4 to about 6. An alternative technique for substantially closing the insertion end of an applicator is by pleating the insertion end. This technique is disclosed in Neilsen et al., U.S. Pat. No. 5,782,793. When an applicator is constructed with more than one layer of material, a single layer may extend into the insertion end in an effort to reduce the force required to expel the contained materials. An example of this is disclosed in Fox et al., U.S. Pat. No. 5,827,214. These collective closures may be of spherical shape, or alternatively tapered shape.

Plastic applicators may be manufactured using any useful technique. Many techniques are known for manufacturing plastic applicators. A representative, non-limiting list of such techniques includes injection-molding, blow-molding, extrusion, formation from one or more sheets (as described above for paper), and the like. Generally, at least the tubular insertion members can be formed through an injection molding process. This process is often used, because it allows the manufacture to balance some key characteristics of the tubular insertion member. Molding inserts and cores can be machined to form a slightly tapered product. For example, the wall thickness around the gripping end is relatively thick to maintain structural stability during the insertion and expulsion steps of use, while the thickness in the insertion end can be minimized to provide flexibility and low expulsion force. Injection molding also enables the manufacture to make uniquely shaped tubular insertion members. As mentioned above, the less sophisticated and/or less expensive techniques, such as extrusion and blow molding can also be employed.

The intrawall conduits described above relating to FIGS. 8-11 can be more easily formed in plastic applicators. The appropriate number and form of channels can be formed through procedures known to those of ordinary skill in the art. In addition to forming the channels during molding, an open channel in the outer surface of an insertion member can be covered with a film or other outer member to enclose the channel.

The lubricant carrier may be attached through adhesives, crimping, or heat sealing, as described briefly, above. It may be fully or partially attached. A partially attached lubricant carrier may be attached at one end, e.g., the insertion end, of the insertion member, it may be attached in circumferential rings, e.g., as shown in FIG. 3), or it may be attached with a longitudinal orientation, e.g., wings extending radially outwards from a line extending along the insertion barrel. The lubricant carrier may be attached after formation and loading of the applicator with the insertable device, during manufacture, or by the user prior to use (while packaged, during the package removal, or after removal from the outer package material).

The applicator of the present invention can be used for the delivery of an object into a mammalian body cavity. Such objects may include suppositories, absorbent devices, and the like, and they may be delivered into body cavities including the mouth, nose, vagina, and rectum. These materials may be in the form of solids, creams, foams, gels, and the like.

Preferably, the applicator is used to deliver intravaginal devices, including catamenial devices, such as tampons, intravaginal collection devices, and interlabial pads; birth control devices such as diaphragms or intrauterine devices (IUDs); compositions in the form of suppositories, such as medicaments, moisturizers, vitamins and minerals, spermicides, and odor controlling agents; medical devices and incontinence devices and vaginal supports such as pessaries; and obstructing devices. Obstructing devices include menstrual collection cups and inflatable or expandable blocking devices.

In use, a woman may place the insertion end into the body cavity orifice, delivering the insertable device into the body cavity by pushing on expulsion member until the insertable device is expelled from the tubular insertion member and withdrawing applicator from the body, leaving insertable device within the body cavity. As mentioned above, in the embodiments of, e.g., FIGS. 3-6, the lubricant carrier may invert during withdrawal, providing a cleansing surface for improved feminine hygiene.

Alternately, a user could pull tubular insertion member onto expulsion member while maintaining expulsion member steady relative the user's body. This substantially eliminates friction between the insertable device and the user's body.

What is claimed is:

1. An applicator for delivering an insertable device into a body cavity comprising
a. a barrel having an insertion end and an-opposed gripper end;
b. a lubricant carrier operatively linked to the barrel; and
c. a fluid lubricant in fluid communication with lubricant carrier;
wherein the barrel is arranged and configured to contain the insertable device.

2. The applicator of claim 1, which further comprises a plunger arranged and configured to be moveable within the barrel to expel the insertable device from the barrel and into a body cavity.

3. The applicator of claim 1, wherein the lubricant carrier is textured.

4. The applicator of claim 1, wherein the lubricant carrier is affixed to the barrel.

5. The applicator of claim 1, wherein the lubricant carrier is affixed to the insertable device.

6. The applicator of claim 4, wherein the lubricant carrier comprises at least one flexible element having an attachment portion that is affixed to the barrel and a distal portion that is capable of extending away from the barrel.

7. The applicator of claim 6, wherein the lubricant carrier comprises a plurality of flexible elements, each flexible element having at least one attachment portion that is affixed to the barrel, and at least one distal portion that is capable of extending away from the barrel.

8. The applicator of claim 7, wherein the at least one attachment portion of at least one of the flexible elements is affixed to the insertion end of the barrel.

9. The applicator of claim 1, wherein a fluid lubricant is disposed on the lubricant carrier.

10. The applicator of claim 1, further comprising at least one reservoir containing a fluid lubricant.

11. The applicator of claim 10, wherein the at least one reservoir is in fluid communication with the lubricant carrier.

12. The applicator of claim 1, wherein the fluid lubricant substantially saturates the lubricant carrier.

13. A kit comprising the applicator of claim 1 and at least one reservoir containing a fluid lubricant and capable of delivering the fluid lubricant to the lubricant carrier.

14. A packaged applicator system comprising
a. an insertable device for delivery into a body cavity contained within a barrel having an insertion end and an opposed gripper end;
b. a lubricant carrier affixed to the barrel; and
c. a fluid lubricant disposed on lubricant carrier.

15. The packaged applicator system of claim 14, further comprising a plunger arranged and configured to be moveable within the barrel to expel the insertable device from the barrel and into a body cavity.

16. The packaged applicator system of claim 14, further comprising an overwrap package including a first package element arranged and configured to isolate the lubricant from the insertable device and a second package element enclosing the applicator.

17. The packaged applicator system of claim 16, wherein the first package element is substantially impervious to moisture.

18. The packaged applicator system of claim 16, wherein the first package element is arranged and configured to sealingly contain a fluid lubricant.

19. A packaged applicator comprising:
a. an insertable device for delivery into a body cavity contained within a barrel having an insertion end and an opposed gripper end;
b. a lubricant carrier affixed to the barrel;
c. a fluid lubricant contained in reservoir in fluid communication with lubricant carrier; and
d. a package element substantially enclosing the barrel, lubricant carrier.

20. The packaged applicator of claim 19, further comprising a plunger arranged and configured to be moveable within the barrel to expel the insertable device from the barrel and into a body cavity.
